(19) United States
(12) Patent Application Publication
(10) Pub. No.: US 2016/0058730 A1
(43) Pub. Date: Mar. 3, 2016

(54) PHARMACEUTICAL COMPOSITIONS OF TERIFLUNOMIDE

(71) Applicant: CADILA HEALTHCARE LIMITED, Ahmedabad (IN)

(72) Inventors: HANDA AJAY KUMAR, Ahmedabad (IN); GUPTA AMIT OMPRAKASH, Ahmedabad (IN); SONI CHIRAG DEEPAKKUMAR, Ahmedabad (IN)

(73) Assignee: CADILA HEALTHCARE LIMITED, Ahmedabad (IN)

(21) Appl. No.: 14/837,195

(22) Filed: Aug. 27, 2015

(30) Foreign Application Priority Data
Aug. 29, 2014 (IN) 2767/MUM/2014

(51) Int. Cl. A61K 31/277; A61K 9/28; A61K 9/20

(52) U.S. Cl. CPC A61K 31/277; A61K 9/2054; A61K 9/2086; A61K 9/2009; A61K 9/2813; A61K 9/2077

(57) ABSTRACT

The present invention relates to stable pharmaceutical compositions of teriflunomide or a pharmaceutically acceptable salt thereof. In particular, the invention relates to the stable pharmaceutical compositions of teriflunomide or a pharmaceutically acceptable salt thereof with colloidal silicon dioxide. The invention also relates to processes for the preparation of such compositions and use thereof for treatment of relapsing forms of multiple sclerosis.

### FIELD OF THE INVENTION

The present invention relates to stable pharmaceutical compositions of teriflunomide or a pharmaceutically acceptable salt thereof. In particular, the invention relates to the stable pharmaceutical compositions of teriflunomide or a pharmaceutically acceptable salt thereof with colloidal silicon dioxide. The invention also relates to processes for the preparation of such compositions and use thereof for treatment of multiple sclerosis.

### BACKGROUND OF THE INVENTION

Teriflunomide is a well-known de novo pyrimidine synthesis inhibitor of the DHO-DH enzyme and is disclosed in U.S. Pat. No. 4,965,276. Chemically, it is (Z)-2-cyano-3-hydroxy-but-2-enoic acid-(4′-trifluoromethylphenyl)-amide having the structural formula I

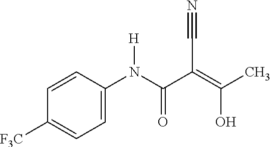 [SMILES: C/C(O)=C(\C#N)C(=O)Nc1ccc(C(F)(F)F)cc1]

It is an active metabolite of leflunomide. Teriflunomide is used to treat relapsing forms of multiple sclerosis. It is not a cure for multiple sclerosis but is thought to work by decreasing certain immune system cells (lymphocytes) which can attack the nerves in brain and spinal cord. This helps to decrease the number of flare-ups (relapses) and may help to slow down physical problems caused by multiple sclerosis.

U.S. Pat. Nos. 5,459,163 and 5,679,709 disclose compositions useful for treating autoimmune diseases in particular lupus erythematosus.

European Patent No. 1381356 B1 discloses the use of Teriflunomide for the manufacture of a medicament for treating multiple sclerosis wherein said medicament is administered orally.

PCT publication No. WO 2007/118684 discloses leflunomide containing solid pharmaceutical compositions including an organic or inorganic acid characterized by improved stability. Said compositions show a slighter decomposition of Leflunomide to Teriflunomide than in commercial Arava® tablets.

Earlier, it was observed that during storage teriflunomide was converted to one degradant, which is 2-cyano-N-(4-trifluoromethyl-phenyl)-acetamide and has the structure illustrated in Formula II:

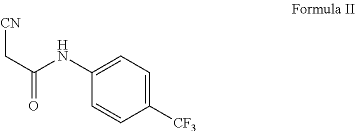 [SMILES: N#CCC(=O)Nc1ccc(C(F)(F)F)cc1]

At room temperature storage 2-cyano-N-(4-trifluoromethyl-phenyl)-acetamide levels of up to 0.2% are reached in the solid pharmaceutical formulation after 12 month storage [Teriflunomide 7 mg tablets, Al/PVC blisters, storage at 25±2° C. and 60% relative humidity {RH}]. A further degradant could be 4-trifluoromethyl-aniline.

U.S. Publication No. 2012/0208880 discloses a stable solid pharmaceutical composition of teriflunomide which is free of colloidal silicon dioxide.

The prior art reference emphasizes on not using colloidal silicon dioxide for producing storage stable pharmaceutical composition of teriflunomide.

Hence, there still remains a need for alternate pharmaceutical compositions comprising teriflunomide which is storage stable and devoid of degradation impurities.

### SUMMARY OF THE INVENTION

In one general aspect there is provided a stable pharmaceutical composition comprising teriflunomide or a pharmaceutically acceptable salt thereof, colloidal silicon dioxide and one or more pharmaceutically acceptable excipients.

In another general aspect there is provided a stable pharmaceutical composition comprising teriflunomide or a pharmaceutically acceptable salt thereof, colloidal silicon dioxide and one or more pharmaceutically acceptable excipients, wherein teriflunomide and colloidal silicon dioxide are not in direct physical contact.

In another general aspect there is provided a stable pharmaceutical composition comprising teriflunomide or a pharmaceutically acceptable salt thereof, colloidal silicon dioxide and one or more pharmaceutically acceptable excipients, wherein the pH of the pharmaceutical composition is not less than about 3.

In another general aspect there is provided a stable pharmaceutical composition comprising teriflunomide or a pharmaceutically acceptable salts thereof, colloidal silicon dioxide and one or more pharmaceutical excipients, wherein the pharmaceutical composition contains no more than about 1% by weight of 2-cyano-N-(4-trifluoromethylphenyl) acetamide after being stored at 40° C. and 75% relative humidity for about 6 months.

In another general aspect there is provided a stable pharmaceutical composition comprising teriflunomide or a pharmaceutically acceptable salts thereof, colloidal silicon dioxide and one or more pharmaceutical excipients, wherein it retains at least 90% of the potency of teriflunomide or a pharmaceutically acceptable salt thereof after storing the composition at 40° C. and 75% relative humidity for at least three months.

In another general aspect there is provided a process for preparing a pharmaceutical composition of teriflunomide or a pharmaceutically acceptable salts thereof, wherein the process comprising the steps of preparing granules of teriflunomide or a pharmaceutically acceptable salts thereof, preparing barrier coated granules of colloidal silicon dioxide, mixing both the granules and processing the mixture to provide a final dosage form.

In another general aspect there is provided a method of treating multiple sclerosis in a patient comprising administering to said subject a pharmaceutical composition comprising teriflunomide or a pharmaceutically acceptable salt thereof, colloidal silicon dioxide and one or more pharmaceutically acceptable excipients.

The details of one or more embodiments of the present invention are set forth in the description below. Other features, objects and advantages of the invention will be apparent from the description.

### DETAILED DESCRIPTION OF THE INVENTION

We have surprisingly found that stable compositions of teriflunomide can be prepared using colloidal silicon dioxide and such compositions have similar stability profile to Aubagio®, the marketed dosage form of teriflunomide in USA.

The inventors have developed a stable pharmaceutical composition of teriflunomide using colloidal silicon dioxide. In particular, the inventors have developed pharmaceutical compositions, wherein teriflunomide and colloidal silicon dioxide are not in direct physical contact.

Moreover, such compositions are also stable and may retain at least 90% of the potency of teriflunomide or a pharmaceutically acceptable salt thereof after storing the composition at 40° C. and 75% relative humidity for at least three months.

As used herein, the term “teriflunomide” is used in broad sense to include not only the teriflunomide per se but also its pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs thereof, and also its various crystalline and amorphous forms. In one embodiment of the present invention, teriflunomide may have particle size D₅₀ less than 50 μm.

The term “colloidal silicon dioxide” refers to submicroscopic fumed silica, also known as pyrogenic silica. It is a non-crystalline, fine grain, low density and high surface area silica. Primary particle size is from 5 nm to 50 nm. The particles are non-porous and have a surface from 50 m²/g to 600 m²/g. It may cover co-processed excipients containing colloidal silicon dioxide like silicified microcrystalline cellulose. The amount of colloidal silicon dioxide present in the pharmaceutical composition of teriflunomide ranges from about 0.1% to about 10% by total weight of the composition.

The term “Degradant” refers to any drug-based materials generated after the preparation of the unit dosage form. Analysis of impurities and degradant is done using reverse phase HPLC techniques on extracted samples as is known in the art.

The phrase “pharmaceutically acceptable” is employed herein to refer to those compounds, materials, compositions; and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The term “direct physical contact” refers to the absence of a barrier layer between components or adjacent compartments of a unit dosage form.

Embodiments of the present invention relate to pharmaceutical compositions of teriflunomide, colloidal silicon dioxide and one or more pharmaceutically acceptable excipients.

The pharmaceutical composition of the present invention refers to pharmaceutical compositions which can be formulated into powder, granule, fine granule/micro-granules, pellets, wafers, tablet or capsule.

In one embodiment, the pharmaceutical composition may comprise teriflunomide or a pharmaceutically acceptable salt thereof, colloidal silicon dioxide and one or more pharmaceutically acceptable excipients.

In another embodiment, the pharmaceutical composition may comprise teriflunomide or a pharmaceutically acceptable salt thereof, colloidal silicon dioxide and one or more pharmaceutically acceptable excipients, wherein teriflunomide and colloidal silicon dioxide are not in direct physical contact.

In another embodiment, the pharmaceutical composition may comprise teriflunomide or a pharmaceutically acceptable salt thereof, colloidal silicon dioxide and one or more pharmaceutically acceptable excipients, wherein the composition is devoid of an acidic reacting compound.

Examples of an acidic reacting compound include but not limited to, citric acid, acetic acid, glycolic acid, lactic acid, pyruvic acid, malonic acid, succinic acid, glutaric acid, fumaric acid, malic acid, tartaric acid, ascorbic acid, maleic acid, hydroxymaleic acid, benzoic acid, hydroxybenzoic acid, phenylacetic acid, cinnamic acid, salicyclic acid, 2-phenoxybenzoic acid, p-toluenesulfonic acid and sulfonic acids such as methanesulfonic acid and 2-hydroxyethanesulfonic acid or a mixture of one or more of said acidic reacting compound.

In another embodiment, the pharmaceutical composition may comprise teriflunomide or a pharmaceutically acceptable salt thereof, colloidal silicon dioxide and one or more pharmaceutically acceptable excipients, wherein teriflunomide and colloidal silicon dioxide are separated by a barrier layer.

In another embodiment, the pharmaceutical composition may comprise teriflunomide or a pharmaceutically acceptable salt thereof, colloidal silicon dioxide and one or more pharmaceutically acceptable excipients, wherein teriflunomide and colloidal silicon dioxide are separated by a barrier layer and colloidal silicon dioxide is a part of outer coating membrane surrounding the core tablet of teriflunomide of a pharmaceutically acceptable salt thereof.

A barrier layer may comprise a water-soluble, pH independent film. Materials that can be used for readily soluble films are well known in the art and may include cellulose derivatives such as hydroxypropylmethyl cellulose, hydroxypropyl cellulose, methacrylic polymers, amino-alkylmethacrylate copolymers (e.g. Eudragit™ E), and polyvinyl alcohol (PVA). A plasticizer (e.g., triacetin, diethyl phthalate, tributyl sebacate or polyethylene glycol) may also be included. Colorants such as titanium dioxide, iron oxide based colorants or others may be included. In one embodiment the barrier layer comprises a non-toxic edible polymer, edible pigment particles, an edible polymer plasticizer, and a surfactant. The thickness of the barrier layer can vary over a wide range, but is generally in the range 20 to 3,000 microns, such as on the order of about 25 to 250 microns.

In another embodiment, the pharmaceutical composition may comprise teriflunomide or a pharmaceutically acceptable salt thereof, colloidal silicon dioxide and one or more pharmaceutically acceptable excipients, wherein colloidal silicon dioxide is coated on a barrier coated core comprising teriflunomide.

In another embodiment, a multilayer pharmaceutical composition may comprise teriflunomide or a pharmaceutically acceptable salt thereof, colloidal silicon dioxide and one or more pharmaceutically acceptable excipients, wherein colloidal silicon dioxide and teriflunomide are separated by a barrier layer.

In another embodiment, the pharmaceutical composition may comprise granules of teriflunomide or a pharmaceutically acceptable salt thereof, granules of barrier coated colloidal silicon dioxide and one or more pharmaceutically acceptable excipients.

In another embodiment, the stable pharmaceutical composition may comprise teriflunomide or a pharmaceutically acceptable salt thereof, colloidal silicon dioxide and one or more pharmaceutically acceptable excipients, wherein the pH of the pharmaceutical composition is not less than 3.

In another embodiment, the pharmaceutical composition may comprise teriflunomide or a pharmaceutically acceptable salt thereof, colloidal silicon dioxide and one or more pharmaceutically acceptable excipients, wherein the amount of colloidal silicon dioxide is in the range of about 0.1 to 10% by weight, preferably 0.2 to 0.5% by weight of the total composition.

In another embodiment, the pharmaceutical composition may comprise teriflunomide or a pharmaceutically acceptable salt thereof, colloidal silicon dioxide and one or more pharmaceutically acceptable excipients, wherein the amount of teriflunomide is in the range of about 1 to 30% by weight of the total composition.

In another embodiment, the pharmaceutical composition may comprise teriflunomide or a pharmaceutically acceptable salt thereof, colloidal silicon dioxide, at least one alkalizer and one or more pharmaceutically acceptable excipients.

In another embodiment, the stable pharmaceutical composition may comprise teriflunomide or a pharmaceutically acceptable salt thereof, colloidal silicon dioxide and one or more pharmaceutical excipients, wherein the pharmaceutical composition contains no more than about 1% by weight of 2-cyano-N-(4-trifluoromethylphenyl)acetamide after being stored at 40° C. and 75% relative humidity for about 6 months.

In another embodiment, the stable pharmaceutical composition may comprise teriflunomide or a pharmaceutically acceptable salt thereof, colloidal silicon dioxide and one or more pharmaceutical excipients, wherein the pharmaceutical composition contains no more than about 0.3% by weight of 2-cyano-N-(4-trifluoromethylphenyl) acetamide after being stored at 40° C. and 75% relative humidity for about 6 months.

The pharmaceutically acceptable excipients for use in the pharmaceutical composition of teriflunomide may include one or more diluents, fillers/bulking agents, binders, disintegrants, surfactants, lubricants, glidants, sweeteners/taste masking agents, colorants and flavors.

Suitable diluents/fillers or bulking agents include, but are not limited to, microcrystalline cellulose, di- or tri-basic calcium phosphate, crystalline cellulose, powdered cellulose, calcium carbonate, calcium sulphate, magnesium silicate, magnesium trisilicate, magnesium aluminium metasilicate (Neusilin), kaolin, starch, starch derivatives, magnesium carbonate, magnesium oxide, co-processed insoluble excipients and sugars such as sucrose, maltose, dextrose, lactose and the like.

Suitable binders include, but are not limited to, hydroxyethyl cellulose, hydroxypropyl cellulose, hydroxypropyl methylcellulose, carbomers, dextrin, ethyl cellulose, methylcellulose, shellac, zein, gelatin, gum arabic, polymethacrylates, polyvinyl pyrrolidone, polyvinyl alcohol, polyethylene glycol, carrageenan, polyethylene oxide, waxes, pullulan, agar, tragacanth, veegum, pregelatinized starch, sodium alginate, gums and the like.

Suitable disintegrants include, but are not limited to, Veegum (highly refined isomorphous silicate), crospovidone, cellulose, kaolin, crosslinked carboxy methyl cellulose (e.g., AcDiSol), microcrystalline cellulose (e.g., Avicel PH101 & PH102), crosslinked polyvinyl pyrrolidone (e.g., Kollidon CL), and mixtures thereof. Preferred disintegrants among these disintegrants include crosslinked carboxy methyl cellulose (e.g., AcDiSol), microcrystalline cellulose (e.g., Avicel PH101 & PH102), crosslinked polyvinyl pyrrolidone (e.g., Kollidon CL), and mixtures thereof. The amount of disintegrant in the pharmaceutical composition ranges from about 0.5% to about 40% by total weight of the composition. In the preferred embodiment disintegrant is microcrystalline cellulose in an amount of not less than 20% by weight of the composition.

Suitable alkalizers include, but are not limited to, organic and inorganic basic compounds of a wide range of aqueous solubility, molecular weights and mixtures thereof. Representative examples include ammonium hydroxide, meglumine, tromethamine, alkali metal salts, alkaline earth metal salts such as magnesium oxide, magnesium hydroxide, calcium hydroxide, sodium hydroxide, potassium hydroxide, aluminum hydroxide, potassium carbonate, sodium bicarbonate or mixtures thereof.

Suitable surfactants include, but are not limited to, anionic, cationic, non-ionic or amphoteric surfactants or those known to the person skilled in the art. Anionic surfactants include sodium lauryl sulfate, sodium dodecanesulfonate, sodium oleyl sulfate, and sodium laurate mixed with stearates and talc. Cationic surfactants include benzalkonium chlorides and alkyltrimethylammonium bromides. Neutral surfactants include glyceryl monooleate, polyoxyethylene sorbitan fatty acid esters, polyvinyl alcohol, and sorbitan esters. The amount of surfactant present in the pharmaceutical composition of teriflunomide a pharmaceutically acceptable salt thereof ranges from about 0.5% to about 25% by total weight of the composition.

Suitable lubricants and glidants include, but are not limited to, stearic acid and its derivatives or esters like sodium stearate, magnesium stearate and calcium stearate and the corresponding esters such as sodium stearyl fumarate and talc. The amount of lubricant present in the pharmaceutical composition of teriflunomide ranges from about 0.1% to about 5% by total weight of the composition.

Suitable taste masking agents include, but are not limited to, one or more of polymers, surfactants, sweeteners and flavors. Examples of polymers include one or more of cellulose acetate, polymethacrylates, cellulose derivatives such as hydroxypropylmethyl cellulose, hydroxypropyl cellulose, hydroxylethyl cellulose; and the like. Examples of sweeteners include but not limiting to one or more of aspartame, saccharin, sucralose, glycyrrhizin; and the like.

Suitable sweeteners include, but are not limited to, saccharides such as aspartame, sugar derivatives. Other examples of sweeteners comprise sodium saccharin; aspartame; sugarless sweeteners, hydrogenated starch hydrolysates, alone or in combination.

Suitable flavors include, but are not limited to, cinnamon, wintergreen, eucalyptus, spearmint, peppermint, menthol, anise as well as fruit flavors such as apple, pear, peach, vanilla, strawberry, cherry, apricot, orange, watermelon, banana and the like; bean-derived flavors, such as coffee, cocoa and the like or mixtures thereof.

In another embodiment, the pharmaceutical composition of teriflunomide or a pharmaceutically acceptable salt thereof may be prepared by any suitable method known in the art such as direct compression, dry or wet granulation, fluidized bed granulation, melt extrusion, melt granulation, spray coating, freeze drying, spray drying and solution evaporation.

In another embodiment, the pharmaceutical composition of teriflunomide or a pharmaceutically acceptable salt thereof may be prepared by:
(1) premixing the teriflunomide or a pharmaceutically acceptable salt thereof and the main portion of the excipients including the binder to obtain a pre-mixture;
(2) granulating the pre-mixture by adding the granulation liquid, preferably water,
(3) drying the teriflunomide granules in a fluidized bed dryer or a drying oven;
(4) optionally dry sieving of the dried teriflunomide granules;
(5) preparing granules of colloidal silicon dioxide;
(6) applying the solution of barrier coating on the granules of colloidal silicon dioxide;
(7) mixing the granules of teriflunomide and barrier coated granules of colloidal silicon dioxide with the remaining excipients like a glidant and a lubricant to obtain the final mixture;
(8) tableting the final mixture by compressing it on a suitable tablet press to produce tablets cores; and
(9) optionally film-coating of the tablet cores with a film coat.

In another embodiment, the pharmaceutical composition of teriflunomide or a pharmaceutically acceptable salt thereof may be prepared by:
(1) preparing core tablets of teriflunomide or a pharmaceutically acceptable salt thereof;
(2) coating the core tablets with HPMC based barrier coating; and
(3) overcoating the seal coated tablets with colloidal silicon dioxide containing coating material.

In another embodiment, the pharmaceutical composition of teriflunomide or a pharmaceutically acceptable salt thereof may be prepared by:
(1) preparing granules of teriflunomide or a pharmaceutically acceptable salt thereof;
(2) preparing granules of colloidal silicon dioxide;
(3) preparing granules of barrier layer polymer and
(4) compressing these three types of granules in a multilayer tablet, wherein barrier layer portion is sandwiched between teriflunomide layer and colloidal silicon dioxide layer.

In another embodiment, the pharmaceutical composition of teriflunomide or a pharmaceutically acceptable salt thereof may be prepared by:
(1) preparing granules of teriflunomide or a pharmaceutically acceptable salt thereof;
(2) coating the teriflunomide granules with HPMC based barrier coating;
(3) mixing the coated granules with colloidal silicon dioxide and
(4) compressing the mixture of coated granules and colloidal silicon dioxide in a tablet.

In another embodiment, the pharmaceutical composition of teriflunomide or a pharmaceutically acceptable salt thereof may be prepared by:
(1) preparing granules of teriflunomide or a pharmaceutically acceptable salt thereof;
(2) coating the teriflunomide granules with HPMC based barrier coating;
(3) mixing the coated granules with silicified microcrystalline cellulose and magnesium stearate; and
(4) compressing the mixture of coated granules and silicified microcrystalline cellulose in a tablet.

The invention further provides a method of treating relapsing forms of multiple sclerosis in patient comprising administering to said subject a pharmaceutical composition comprises teriflunomide or a pharmaceutically acceptable salt thereof, colloidal silicon dioxide and one or more pharmaceutical excipients.

The pharmaceutical compositions described herein have a similar stability profile to Aubagio®, marketed form of teriflunomide tablets.

The invention is further illustrated by the following examples which are provided to be exemplary of the invention and do not limit the scope of the invention.

### Example 1

TABLE 1

| | | Quantity |
|---|---|---|
| Sr. No. | Ingredients | (% w/w) |
| 1 | Teriflunomide | 9.03 |
| 2 | Lactose monohydrate | 46.45 |
| 3 | Microcrystalline cellulose | 9.68 |
| 4 | Maize starch | 19.19 |
| 5 | Hydroxypropylcellulose | 3.87 |
| 6 | Sodium starch glycolate | 4.84 |
| 7 | Hypromellose 3 cps | 3.23 |
| 8 | Purified water | q.s. |

Lubrication

| | | |
|---|---|---|
| 9 | Magnesium stearate | 0.48 |

Film Coating

| | | |
|---|---|---|
| 10 | Opadry containing 10% w/w colloidal | 3.23 |
| | silicon dioxide | |
| 11 | Purified water | q.s. |

### Procedure:

Mixture of teriflunomide, microcrystalline cellulose, lactose, maize starch, hydroxypropylcellulose and sodium starch glycolate was prepared. Solution of Hydroxypropylmethyl cellulose (HPMC) in water was prepared. The mixture containing teriflunomide was granulated with HPMC binder solution. These granules were milled to achieve desired size of granules, which were then subjected to lubrication. Granules were then compressed into tablets. Core tablets were coated with a coating preparation having 10% colloidal silicon dioxide to get a final composition.

TABLE 1a
Stability data of Example 1 after storing the final composition in
alu-alu blister with a desiccant at 40° C. and 75% RH

| Impurity | 6 Months |
|---|---|
| 2-cyano-N-[4- | 0.06 |
| (trifluoromethyl)phenyl]acetamide | |
| 4-(Terifluromethyl aniline) | 0.006 |
| Single unknown | 0.11 |
| Total impurity | 0.17 |

### Example 2

TABLE 2

| | | |
|---|---|---|
| | | Quantity |
| Sr. No. | Ingredients | (% w/w) |

Intragranular

| | | |
|---|---|---|
| 1 | Teriflunomide | 9.24 |
| 2 | Microcrystalline cellulose | 3.96 |
| 3 | Lactose monohydrate | 47.52 |
| 4 | Maize starch | 19.64 |
| 5 | Hydroxypropyl methylcellulose | 4.62 |
| 6 | Sodium starch glycolate | 4.95 |
| 7 | Purified water | q.s. |

Extragranular

| | | |
|---|---|---|
| 8 | Silicified Microcrystalline cellulose | 6.60 |
| 9 | Magnesium stearate | 0.50 |

Film Coating

| | | |
|---|---|---|
| 10 | Opadry II | 2.97 |
| 11 | Purified water | q.s. |

### Procedure:

Mixture of teriflunomide, microcrystalline cellulose, lactose monohydrate, maize starch and sodium starch glycolate was prepared. Solution of Hydroxypropyl methylcellulose (HPMC) in purified water was prepared. Mixture containing teriflunomide was granulated with HPMC binder solution. These granules were milled to achieve desired size of granules, which were then mixed with silicified microcrystalline cellulose and magnesium stearate. Final mixture was then compressed into tablets. Core tablets were coated with Opadry II coating to get a final composition.

TABLE 2A
Stability data of Example 2 after storing the final composition in
alu-alu blister with a desiccant at 40° C. and 75% RH

| Impurity | Initial | 1 Month | 3 Month |
|---|---|---|---|
| 2-cyano-N-[4- | ND | ND | ND |
| (trifluoromethyl)phenyl]acetamide | | | |
| 4-(Terifluromethyl aniline) | ND | ND | ND |
| Single unknown | BQL | 0.09 | 0.06 |
| Total impurity | 0.00 | 0.09 | 0.06 |

### Example 3

TABLE 3

| | | |
|---|---|---|
| | | Quantity |
| Sr. No. | Ingredients | (% w/w) |

Layer 1

| | | |
|---|---|---|
| 1 | Teriflunomide | 2-20 |
| 2 | Microcrystalline cellulose | 5-15 |
| 3 | Lactose monohydrate | 0-50 |
| 4 | Corn starch | 0-10 |
| 5 | Magnesium stearate | 0.5-2 |
| 6 | Purified water | q.s. |

Layer 2

| | | |
|---|---|---|
| 7 | Microcrystalline cellulose | 5-15 |
| 8 | Lactose monohydrate | 0-50 |
| 9 | Corn starch | 0-10 |
| 10 | Colloidal silicon dioxide | 0-10 |
| 11 | Magnesium stearate | 0.5-2 |
| 12 | Purified water | q.s. |

Barrier layer

| | | |
|---|---|---|
| 13 | Ethyl cellulose | 0-5 |
| 14 | Polyvinylpyrrolidone | 0-5 |
| 15 | Ethanol | q.s. |

Film coating

| | | |
|---|---|---|
| 16 | Opadry II | 2-5 |
| 17 | Purified water | q.s. |

### Procedure:

### Preparation of Layer-1

Mixture of teriflunomide, microcrystalline cellulose and lactose monohydrate is prepared. This blend is granulated with corn starch in water. Granules are dried. The dried granules are lubricated with magnesium stearate.

### Preparation of Layer-2

Mixture of colloidal silicon dioxide with microcrystalline cellulose and lactose monohydrate is prepared. This blend is granulated with corn starch in water. Granules are dried. The dried granules are lubricated with magnesium stearate.

### Preparation of a Barrier Layer

Ethyl cellulose is granulated using solution of polyvinylpyrrolidone in ethanol. Granules are dried.

Above three layers are compressed together to form a trilayer tablet having a barrier layer between layer 1 and layer 2.

### Example 4

TABLE 4

| | | |
|---|---|---|
| | | Quantity |
| Sr. No. | Ingredients | (% w/w) |

Intragranular

| | | |
|---|---|---|
| 1 | Teriflunomide | 5-20 |
| 2 | Microcrystalline cellulose | 0-50 |
| 3 | Lactose monohydrate | 0-50 |
| 4 | Pregelatinized starch | 0-50 |
| 5 | Hydroxypropyl methylcellulose | 2-10 |
| 6 | Croscarmellose sodium | 5-25 |
| 7 | Purified water | q.s. |

Extragranular

| | | |
|---|---|---|
| 8 | Colloidal silicon dioxide | 1-5 |
| 9 | Hydroxypropyl methylcellulose | 0-5 |
| 10 | Magnesium stearate | 0.5-2 |

Film Coating

| | | |
|---|---|---|
| 11 | Opadry II | 2-5 |
| 12 | Purified water | q.s. |

### Procedure:

Mixture of teriflunomide, microcrystalline cellulose, lactose monohydrate, pregelatinized starch and croscarmellose sodium is prepared. Solution of Hydroxypropyl methylcellulose (HPMC) in water is prepared. Mixture containing teriflunomide is granulated with HPMC binder solution. These granules are milled to achieve desired size of granules, which are then mixed with colloidal silicon dioxide, hydroxypropyl methylcellulose and magnesium stearate. Granules are then compressed into tablets. Core tablets are coated with Opadry II coating to get a final composition.

While the present invention has been described in terms of its specific embodiments, certain modifications and equivalents will be apparent to those skilled in the art and are intended to be included within the scope of the present invention.

We claim:

1. A stable pharmaceutical composition comprising teriflunomide or a pharmaceutically acceptable salt thereof, colloidal silicon dioxide and one or more pharmaceutically acceptable excipients.

2. The pharmaceutical composition according to claim 1, wherein the composition has a pH not less than 3.

3. The pharmaceutical composition according to claim 1, wherein teriflunomide and the colloidal silicon dioxide are not in direct physical contact.

4. The pharmaceutical composition according to claim 1, wherein granules of teriflunomide are mixed with barrier coated granules of colloidal silicon dioxide and then compressed into tablets.

5. The pharmaceutical composition according to claim 1, wherein the composition is a multilayer tablet having colloidal silicon dioxide and teriflunomide separated by a barrier layer.

6. The pharmaceutical composition according to claim 1, wherein the colloidal silicon dioxide is coated on a barrier coated core comprising teriflunomide.

7. The pharmaceutical composition according to claim 1, wherein the composition comprises silicified microcrystalline cellulose.

8. The pharmaceutical composition according to claim 7, wherein the silicified microcrystalline cellulose is added extragranularly.

9. The pharmaceutical composition according to claim 1, wherein the pharmaceutical composition contains no more than about 1% by weight of 2-cyano-N-(4-trifluoromethylphenyl) acetamide after being stored at 40° C. and 75% relative humidity for about 6 months.

10. The pharmaceutical composition according to claim 1 retains at least 90% of the potency of teriflunomide or a pharmaceutically acceptable salt thereof after storing the composition at 40° C. and 75% relative humidity for at least three months.

11. A method of treating relapsing forms of multiple sclerosis comprising administering to a human patient in need thereof the pharmaceutical composition according to claim 1.
